(19) United States
(12) Patent Application Publication
(10) Pub. No.: US 2009/0156457 A1
(43) Pub. Date: Jun. 18, 2009

(54) METHODS AND MEANS FOR SCREENING FOR RHOMBOID ACTIVITY

(75) Inventor: Keith H. Ansell, London (GB)

Correspondence Address: BOZICEVIC, FIELD & FRANCIS LLP, 1900 UNIVERSITY AVENUE, SUITE 200, EAST PALO ALTO, CA, 94303, US

(73) Assignee: MEDICAL RESEARCH COUNCIL, London (UK)

(21) Appl. No.: 10/597,140

(22) PCT Filed: Jan. 17, 2005
(86) PCT No.: PCT/GB05/00154

§ 371 Date: Apr. 5, 2007

Related U.S. Application Data
(60) Provisional application No. 60/536,860, filed on Jan. 16, 2004.

(51) Int. Cl. A61K 38/00; G01N 33/53; C07K 2/00; C12N 9/16; C07H 21/04; C12N 15/63; C12N 5/00; C12P 21/06; C12Q 1/00

(52) U.S. Cl. 514/2; 435/7.1; 530/300; 435/196; 536/23.1; 435/320.1; 435/325; 435/68.1; 435/4

(57) ABSTRACT

This invention relates to methods of screening for rhomboid modulating compounds using a substrate polypeptide has a core domain comprising a rhomboid cleavable TMD sequence linked to an upstream tag sequence. The core domain sequence is not susceptible to cleavage by non-rhomboid proteases so products of rhomboid dependent proteolysis products may be detected by determining the presence of the tag sequence. Rhomboid modulating compounds identified by the present methods may be useful in a range of therapeutic applications.

This invention relates to methods of screening for compounds which modulate the activity of rhomboid proteins. Modulatory compounds may be useful in a range of therapeutic applications.

Rhomboids are a conserved family of intermembrane serine proteases which are involved in controlling diverse biological functions (Urban and Freeman Mol Cell. 2003 Jun; 11(6): 1425-34, Urban, S. et al (2002) EMBO J. 21, 4277-4286, Urban, S. et al (2002) Current Biology 12, 1507-1512). Screening methods suitable for use in high throughput formats are an important step in the development of therapeutics which target rhomboids.

Known methods of screening for rhomboid activity lack sensitivity, have a low signal to background ratio and are unsuitable for use in high-throughput formats (Urban et al (2003) supra; WO02/093177). In particular, rhomboid-independent proteolysis leads to sensitivity and background problems and often needs to be suppressed with inhibitors (e.g. batimastat; British Biotech).

Transmembrane proteins, including, for example, EGF, TNFα, TGFα and other EGF receptor ligands, are substrates for metalloproteases (MPs), including ADAM (a disintegrin and metalloprotease) family MPs, such as TACE (tumour necrosis factor α convertase). These enzymes cleave their substrates to release the extracellular domain in a process known as ectodomain shedding. Ectodomain shedding is sensitive to metalloprotease inhibitors such as batimastat, which contain a hydroxamate group that acts as a zinc-binding group. (Pandiella, A. & Massague, J. (1991) J Biol Chem 266, 5769-73, Arribas, J. et al (1997) J Biol Chem 272, 17160-5, Wang, X. et al (2003) Mol Endocrinol 17, 1931-43, Seals, D. F. & Courtneidge, S. A. (2003). Genes Dev 17, 7-30).

No precise consensus has emerged for the cleavage determinant of ADAM family MPs. However a consistent feature is that the cleavage determinant is generally located in a stalk region between the membrane and an initial globular extracellular subdomain (Wang, X. et al (2002) J Biol Chem 277, 50510-9). For TGFα, 14 juxtamembrane residues are sufficient to confer shedding and a lack of secondary structure in the juxtamembrane region may confer susceptibility to sheddases rather than a specific primary sequence motif (Arribas et al. (1997) supra). The cleavage of the extracellular domain of GH binding protein by MP-mediated sheddase has been reported to occur at a position 9 residues outside the transmembrane domain (Wang et al. 2003).

The present inventors have developed improved methods of screening for rhomboid modulators which reduce the problems associated with rhomboid-independent proteolysis.

A first aspect of the invention provides a method for identifying and/or obtaining a modulator of a rhomboid polypeptide, which method comprises:

(a) contacting a rhomboid polypeptide and a substrate polypeptide in the presence of a test compound and one or more non-rhomboid proteases,

wherein said substrate polypeptide comprises a core domain which includes a rhomboid cleavable transmembrane domain (TMD) sequence and a tag sequence, the core domain sequence not being susceptible to cleavage by the one or more non-rhomboid proteases, and;

(b) determining the presence in said medium of a polypeptide fragment comprising said tag sequence.

Cleavage of the substrate polypeptide to generate the fragment may be determined in the presence and absence of test compound. A difference in cleavage in the presence of the test compound relative to the absence of test compound may be indicative of the test compound being a modulator of rhomboid protease activity.

The rhomboid and substrate polypeptides may be contacted under conditions wherein, in the absence of the test compound, the rhomboid polypeptide cleaves the TMD sequence of the substrate polypeptide to produce a polypeptide fragment comprising the tag sequence. The presence of such a fragment in the medium is then detected by means of the tag sequence.

Non-rhomboid proteases may be soluble or membrane bound and may include metalloproteases (MPs) including ADAM metalloproteases, such as TACE.

Non-rhomboid proteases cleave the substrate polypeptide to produce polypeptide fragments. However, whilst rhomboid proteases cleave within the TMD, non-rhomboid proteases cleave outside the core domain (i.e. upstream of the tag sequence) and the proteolytic fragments thus produced lack the tag sequence. The position of the tag within the substrate polypeptide thus allows discrimination between non-rhomboid and rhomboid cleavage events.

The rhomboid polypeptide and the substrate polypeptide are preferably membrane-bound. The polypeptides may be co-expressed within a cell, for example a yeast, insect or mammalian cell, for example a CHO, HeLa or COS cell. The polypeptide fragment is preferably soluble and is secreted into the medium after cleavage.

The core domain is preferably a chimeric sequence which comprises a rhomboid cleavable TMD and a heterogenous tag sequence. The tag sequence may be positioned within the TMD or, more preferably, upstream of the TMD i.e. positioned within the core domain closer to the N terminal or extracellular/luminal domain than the TMD. The tag sequence is preferably an affinity tag, i.e. a heterogeneous peptide sequence which forms one member of a specific binding pair. Polypeptides containing the tag may be detected by determining the binding of the other member of the specific binding pair to the polypeptide. In some preferred embodiments, the tag sequence may form an epitope which is bound by an antibody molecule.

A tag sequence may consist of at least 2, 4, 6, or 8 amino acid residues. A tag sequence may consist of 25 or less, 20 or less, 15 or less or preferably 10 or less amino acid residues.

Various suitable tag sequences are known in the art, including, for example, MRGS(H)₆, DYKDDDDK (FLAG™), T7-, S- (KETAAAKFERQHMDS), poly-Arg (R₅₋₆), poly-His (H₂₋₁₀), poly-Cys (C₄) poly-Phe(F₁₁) poly-Asp(D₅₋₁₆), Strept-tag II (WSHPQFEK), c-myc (EQKLISEEDL), Influenza-HA tag (Murray, P. J. et al (1995) Anal Biochem 229, 170-9), Glu-Glu-Phe tag (Stammers, D. K. et al (1991) FEBS Lett 283, 298-302), Tag.100 (Qiagen; 12 aa tag derived from mammalian MAP kinase 2), Cruz tag 09™ (MKAEFRRQESDR, Santa Cruz Biotechnology Inc.) and Cruz tag 22™ (MRDALDRLDRLA, Santa Cruz Biotechnology Inc.). Known tag sequences are reviewed in Terpe (2003) Appl. Microbiol. Biotechnol. 60 523-533.

In preferred embodiments, a poly-His tag such as MRGS(H)₆ is used.

The tag sequence is preferably positioned adjacent to the rhomboid cleavable TMD sequence within the core domain. For example, the tag sequence may be positioned 10 amino acid residues or less, 5 amino acid residues or less or 2 amino acid residues or less upstream of said TMD. In some embodiments, the tag sequence may be directly linked to said TMD (i.e. immediately upstream of the TMD).

In other embodiments, the tag sequence may be positioned within the TMD. A suitable intramembrane tag sequence may comprise a hydrophobic amino acid sequence.

The substrate polypeptide may comprise any TMD which is proteolytically cleaved by a rhomboid polypeptide. Such TMDs are readily identified using standard techniques.

In some preferred embodiments, a rhomboid cleavable TMD may have a lumenal portion which has the same conformation within the membrane as Spitz (Q01083) residues 140-144 (IASGA) or more preferably Spitz residues 138-144 (ASIASGA), or the equivalent residues in a different rhomboid ligand, such as Gurken (P42287), Keren (AAF63381), Mgm1 (YOR211C), Ccp1 (YKR066C) or mammalian thrombomodulin, for example mouse thrombomodulin (NP_033404), rabbit (Oryctoclagus cuniculus; AAN15931); rat (Rattus norvegicus; NP_113959), cow (Bos Taurus; AAA30785) or human thrombomodulin (AAH533357). Other rhomboid ligands include EGFR ligands, examples of which are shown in Table 2.

The lumenal portion of a rhomboid cleavable TMD may, for example, comprise or consist of Spitz residues 140-144 (IASGA), more preferably Spitz residues 138-144 (ASIASGA), or the equivalent residues in a different rhomboid ligand, such as Gurken, Keren, Mgm1, Ccp1 or thrombomodulin.

In some embodiments, the rhomboid cleavable TMD may be an rhomboid ligand TMD, for example a TMD from a ligand, such as Gurken, Keren, S. cerevisiae polypeptides MGM1/YOR211C and CCP1/YKR066C or mammalian thrombomodulin, or a variant or allele of any of these. In some preferred embodiments, a Spitz TMD may be used.

A variant or allele of a rhomboid ligand may include a polypeptide modified by varying the amino acid sequence of the protein, e.g. by manipulation of the nucleic acid encoding the protein or by altering the protein itself. Such variants of the natural amino acid sequence may involve one or more of insertion, addition, deletion or substitution of one or more amino acids, which may be without fundamentally altering the susceptibility of the polypeptide to proteolytic cleavage by a rhomboid polypeptide.

A TMD from a variant or allele of a rhomboid ligand may have a lumenal portion which has the same conformation within the membrane as the rhomboid ligand. In some embodiments, the TMD of a variant or allele of a rhomboid ligand may consist of a sequence which has the having greater than about 50% sequence identity with the TMD sequence of the rhomboid ligand, greater than about 60%, greater than about 70%, greater than about 80%, greater than about 90%, or greater than about 95%. The sequence may share greater than about 70% similarity with the TMD sequence of the rhomboid ligand, greater than about 80% similarity, greater than about 90% similarity or greater than about 95% similarity.

The substrate polypeptide may comprise a cytoplasmic domain downstream (i.e. towards the C terminal) of the core domain. In some embodiments, the cytoplasmic domain may be the cytoplasmic domain of a rhomboid ligand, for example, a TGFα cytoplasmic domain.

In some preferred embodiments, cytoplasmic domain may be the cytoplasmic domain of thrombomodulin or a variant or allele thereof. When the substrate polypeptide comprises such a cytoplasmic domain, the rhomboid polypeptide is preferably a RHBDL2 polypeptide, as described below.

An variant or allele of the cytoplasmic domain of thrombomodulin may comprise or consist of an amino acid sequence having at least 70%, at least 75%, at least 80%, at least 85%, at least 90% or at least 95% sequence identity with the amino acid sequence of the cytoplasmic domain (residues 540-575) of a mammalian thrombomodulin, for example mouse thrombomodulin (NP_{—}033404) or human thrombomodulin (AAH533357).

Amino acid identity and similarity are generally defined with reference to the algorithm GAP (Genetics Computer Group, Madison, Wiss.). GAP uses the Needleman and Wunsch algorithm to align two complete sequences that maximizes the number of matches and minimizes the number of gaps. Generally, the default parameters are used, with a gap creation penalty=12 and gap extension penalty=4. Use of GAP may be preferred but other algorithms may be used, e.g. BLAST or TBLASTN (which use the method of Altschul et al. (1990) J. Mol. Biol. 215: 405-410), FASTA (which uses the method of Pearson and Lipman (1988) PNAS USA 85: 2444-2448),or the Smith-Waterman algorithm (Smith and Waterman (1981) J. Mol Biol. 147: 195-197), generally employing default parameters.

Similarity allows for “conservative variation”, i.e. substitution of one hydrophobic residue such as isoleucine, valine, leucine or methionine for another, or the substitution of one polar residue for another, such as arginine for lysine, glutamic for aspartic acid, or glutamine for asparagine. Particular amino acid sequence variants or alleles may differ from a known sequence as described herein by insertion, addition, substitution or deletion of 1 amino acid, 2, 3, 4, 5-10, 10-20 20-30, 30-50, or more than 50 amino acids.

Sequence identity and similarity are generally determined over the full-length of the sequence unless context dictates otherwise.

The substrate polypeptide may also comprise an extracellular domain upstream (i.e. towards the N terminal) of the core domain. The extracellular domain may comprise a detectable label. Suitable detectable labels include fluorescent proteins such as green fluorescent protein (GFP), luciferase, alkaline phosphatase and red, yellow, cyan and enhanced versions thereof. Other suitable labels include β-galactosidase, β-lactamase and β-glucuronidase. These labels allow convenient detection of the soluble cleaved product and are particularly useful in automated assays.

In some preferred embodiments, the detectable label is secreted alkaline phosphatase. The presence of a secreted alkaline phosphatase label may be detected by conventional techniques. For example, SEAP may be detected using a chemiluminescent substrate CSPD® (Tropix, Bedford, Mass., USA) (Bronstein, I. et al. (1994). Anal Biochem 219, 169-81) Fluorogenic substrates e.g. MUP, (Molecular Probes) may also be used, although these are less sensitive. Standard alkaline phosphatase substrates, such as p-nitrophenyl phosphate, have still lower sensitivity.

The extracellular domain may also comprise an N terminal signal sequence which directs the secretion of the cleaved polypeptide fragment from the host cell, for example a secreted alkaline phosphatase signal sequence.

A polypeptide substrate may be a chimeric polypeptide comprising sequence from two or more rhomboid ligands along with the tag sequence, for example a chimeric ligand may comprise the transmembrane domain of a first rhomboid ligand in a core domain adjacent to a heterogeneous tag sequence and the intracellular and/or extracellular domains of a second rhomboid ligand.

In some embodiments, a chimeric ligand may comprise a cytoplasmic domain from a first rhomboid ligand and a rhomboid cleavable TMD from a second rhomboid ligand, in addition to a heterogeneous tag.

A suitable rhomboid polypeptide for use in the present methods may have a sequence shown in Table 1.

For example, the rhomboid polypeptide may be selected from the group consisting of Drosophila Rhomboid 1, Drosophila Rhomboid 2, Drosophila Rhomboid 3, Drosophila Rhomboid 4, Human RHBDL-1, Human RHBDL-2 and Human RHBDL-3, E. coli glgG, B. subtilis ypqP, P. stuartii A55862 gene product, P. aeruginosa B83259 gene product, S. cervisiae YGR101w and S. cervisiae YPL246c. Other suitable rhomboids may be identified using conventional database searching methods.

In preferred embodiments, the rhomboid polypeptide is selected from Human RHBDL-1, Human RHBDL-2 and Human RHBDL-3.

Rhomboid polypeptides preferably comprise catalytic residues R152, G215, S217 and H281, more preferably catalytic residues W151, R152, N169, G215, S217 and H281. The presence of these conserved residues may be used to identify rhomboid polypeptides.

Preferably, a rhomboid polypeptide comprises at least 5 TMDs, with residues N169, S217 and H281 each occurring in different TMD at about the same level in the lipid membrane bilayer. Preferably, a rhomboid polypeptide also comprises a GxSG motif, as described above.

Rhomboid amino acid residues are described herein with reference to their position in the Drosophila Rhomboid-1 sequence. It will be appreciated that the equivalent residues in other rhomboid polypeptides may have a different position and number, because of differences in the amino acid sequence of each polypeptide. These differences may occur, for example, through variations in the length of the N terminal domain. Equivalent residues in Rhomboid polypeptides are easily recognisable by their overall sequence context and by their positions with respect to the rhomboid TMDs.

Rhomboid polypeptides are also characterised by the presence of a rhomboid homology domain, as defined by the PFAM protein structure annotation project (Bateman A. et al (2000) The Pfam Protein Families Database Nucl. Acid. Res. 28 263-266). The Pfam rhomboid homology domain is built from a Hidden Markov Model (HMM) using 26 rhomboid sequences as a seed. The Pfam ‘rhomboid’ domain has the pfam specific accession number PF01694.

The rhomboid polypeptide may comprise an ER (endoplasmic reticulum) retention signal. The KDEL ER retention signal is not found in natural rhomboid polypeptides and directs the expressed rhomboid polypeptide to be retained in the ER (endoplasmic reticulum) rather than the Golgi apparatus.

The term “heterologous” may be used to indicate that the nucleic acid sequence in question has been introduced into a nucleic acid construct, vector or cell using genetic engineering, i.e. by human intervention, and is not naturally associated with the nucleic acid sequence of the construct, vector or cell.

Polypeptide fragments which retain the activity of the full-length protein may be generated and used in the methods described herein.

The presence in the medium of rhomboid-cleaved polypeptide fragments comprising the tag sequence may be determined by any convenient technique, for example, Western blotting, capture ELISA, affinity chromatography or other chromatographic method or methods followed by SDS PAGE and/or reporter assay.

In some preferred embodiments, the presence of the soluble polypeptide fragment in the medium may be determined by;
(a) contacting the medium with a specific binding member which binds to the tag sequence, and
(b) determining binding of the soluble polypeptide fragment to the specific binding member.

Suitable specific binding members include an antibody molecule which binds to the tag sequence or an immobilised metal chelate, which binds, for example, to a polyHis tag.

Binding of specific binding members, such as antibody molecules, may be determined by any appropriate means.

Detection of individual label molecules is one possibility. For example, the binding of the polypeptide fragment to the specific binding member may be determined by detecting the level or amount of bound label.

The label may directly or indirectly generate a detectable, and preferably measurable, signal. The level or amount of said label bound to the specific binding member may, for example, be determined by contacting the label with a substrate which reacts with the label to produce a signal.

In some preferred embodiments, the substrate reacts with the label to produce light. For example, the reaction of the label and the substrate may produce luminescence. The subsequent light emission may be measured, for example using a luminometer.

The detectable label may be linked to the specific binding pair member or more preferably to the polypeptide fragment by a direct or indirect, covalent, e.g. via a peptide bond, or non-covalent linkage. Suitable labels may include a fluorophore such as FITC or rhodamine, a radioisotope, or a non-isotopic-labelling reagent such as biotin or digoxigenin; polypeptides containing biotin may be detected using “detection reagents” such as avidin conjugated to any desirable label such as a fluorochrome. In preferred embodiments, a detectable polypeptide label such as green fluorescent protein (GFP), luciferase or alkaline phosphatase may be used. A polypeptide label is preferably comprised within the extracellular domain of the substrate polypeptide.

In some embodiments, the binding of an antibody or other specific binding pair member to a tag-containing polypeptide fragment may be detected using a second antibody. The second antibody may bind to the specific binding pair member (e.g. the first antibody), or may bind to a different region of the same polypeptide fragment, for example in a sandwich assay. Depending on the assay format employed, the second antibody may be immobilised or labelled with a detectable label.

The mode of determining binding to the specific binding member is not a feature of the present invention and those skilled in the art are able to choose a suitable mode according to their preference and general knowledge.

Specific binding pair members, such as antibody molecules, which bind specifically to a tag sequence may be produced using techniques which are conventional in the art. Many suitable specific binding pair members are available commercially (for example, RGS/His tag antibody (Qiagen), Tetra, penta- and hexa- his antibodies (Qiagen), Tag.100 antibody (Qiagen), HA tag antibody (Santa Cruz Biotechnology Inc.), 9E10 antibody against c-myc tag (Santa Cruz Biotechnology Inc), Cruz tag antibodies (Santa Cruz Biotechnology Inc.) and Anti-FLAG tag antibody (Sigma Aldrich)).

A specific binding member, such as an antibody, for use in a method described herein may be immobilised or non-immobilised i.e. free in solution.

An antibody or other specific binding pair member may be immobilised, for example, by attachment to an insoluble support. The support may be in particulate or solid form and may include a plate, a test tube, beads, a ball, a filter or a membrane. An antibody may, for example, be fixed to an insoluble support that is suitable for use in affinity chromatography. Methods for fixing antibodies to insoluble supports are known to those skilled in the art.

A convenient way of producing rhomboid and substrate polypeptides for use in methods described herein is to express nucleic acid encoding them, by use of the nucleic acid in an expression system. This may conveniently be achieved by growing a host cell in culture, containing one or more expression vectors, under appropriate conditions that cause or allow expression of the polypeptides e.g. in eukaryotic cells such as COS or CHO cells or in prokaryotic cells such as E. coli.

The amount of test substance or compound which may be used in a method described herein will normally be determined by trial and error depending upon the type of compound used. Typically, from about 0.01 nM to 40 μM concentrations of putative inhibitor compound may be used, for example from 1 nM to 40 μM. When cell-based assays are employed, the test substance or compound is desirably membrane permeable in order to access the Rhomboid polypeptide.

Test compounds may be natural or synthetic chemical compounds used in drug screening programmes. Extracts of plants which contain several characterised or uncharacterised components may also be used.

Combinatorial library technology (Schultz, J S (1996) Biotechnol. Prog. 12:729-743) provides an efficient way of testing a potentially vast number of different substances for ability to modulate activity of a polypeptide. Prior to or as well as being screened for modulation of activity, test substances may be screened for ability to interact with the Rhomboid polypeptide, e.g. in a yeast two-hybrid system (which requires that both the polypeptide and the test substance can be expressed in yeast from encoding nucleic acid). This may be used as a coarse screen prior to testing a substance for actual ability to modulate Rhomboid activity.

One class of putative inhibitor compounds can be derived from a Rhomboid polypeptide and/or a rhomboid ligand TMD. Membrane permeable peptide fragments of from 5 to 40 amino acids, for example, from 6 to 10 amino acids may be tested for their ability to disrupt such interaction or activity. Especially preferred peptide fragments comprise residues 141 to 144 (ASGA) of the Spitz protein, residues 140-144 (IASGA) or residues 138-144 (ASIAGA), or the equivalent regions of other rhomboid ligands.

The inhibitory properties of a peptide fragment as described above may be increased by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and J. Bond Oxford University Press 2001). Two compounds TPCK and 3, 4-DCI have been shown to inhibit Rhomboid activity. Although these compounds are broad-spectrum serine protease inhibitors, they represent examples of lead compounds for the rational design of specific Rhomboid inhibitors.

Other candidate inhibitor compounds may be based on modelling the 3-dimensional structure of a polypeptide or peptide fragment and using rational drug design to provide potential inhibitor compounds with particular molecular shape, size and charge characteristics. Suitable techniques are well known in the art and are described in more detail below.

A method as described herein may comprise the step of identifying a test compound as an agent which modulates rhomboid activity, for example by determining an increase or decrease in the amount of rhomboid directed substrate polypeptide cleavage in the presence relative to the absence of the compound. The compound may be an inhibitor (antagonist) or enhancer (agonist) rhomboid directed substrate polypeptide cleavage

Following identification of a compound that modulates rhomboid activity, the compound may be investigated further, in particular for its ability to modulate one or more rhomboid-mediated cellular activities. For example, a method may further comprise the step of determining the ability of said test compound to inhibit the infectivity or virulence of a microbial pathogen. This may, for example, comprise determining the expression of toxic virulence factors in the presence and absence of test compound. A microbial pathogen may include yeasts and pathogenic bacteria such as Providencia stuartii, E. coli 0157 and Pseudomonas aeruginosa.

A compound identified as a rhomboid modulator may be isolated and/or purified, or alternatively it may be synthesised using conventional techniques of recombinant expression or chemical synthesised. The compound may be manufactured and/or used in preparation, i.e. manufacture or formulation, of a composition such as a medicament, pharmaceutical composition or drug. These may be administered to individuals for the treatment of disorders as described below. Methods of the invention may thus comprise formulating said test compound in a pharmaceutical composition with a pharmaceutically acceptable excipient, vehicle or carrier for therapeutic application, as discussed further below.

A method of making a pharmaceutical composition may comprise,

identifying a compound as a modulator of Rhomboid activity using a method described herein,

synthesising, preparing or isolating said modulator and,

admixing the modulator with a pharmaceutically acceptable excipient, vehicle or carrier, and optionally other ingredients to formulate or produce said composition; and, optionally,

determining the activity of a Rhomboid polypeptide as described herein in the presence of said composition.

Compounds identified as rhomboid modulators may be modified to optimise activity or other properties such as increased half-life or reduced side effects upon administration to an individual.

The modification of a known pharmacologically active compound to improve its pharmaceutical properties is a known approach to the development of pharmaceuticals based on a “lead” compound. This might be desirable where the active compound is difficult or expensive to synthesise or where it is unsuitable for a particular method of administration, e.g. peptides are not well suited as active agents for oral compositions as they tend to be quickly degraded by proteases in the alimentary canal. The design, synthesis and testing of modified active compounds, including mimetics, may be used to avoid randomly screening large number of molecules for a target property. Whilst TPCK and 3, 4-DCI have been shown to inhibit Rhomboid, these compounds lack specificity and so are liable to produce undesirable side-effects, if used therapeutically. They may however represent “lead” compounds for the development of mimetics with improved specificity.

There are several steps commonly taken in modifying a compound such as TPCK, 3, 4-DCI, or Spitz transmembrane fragments, which has a given target property. Firstly, the particular parts of the compound that are critical and/or important in determining the target property are determined. In the case of a peptide, this can be done by systematically varying the amino acid residues in the peptide, e.g. by substituting each residue in turn.

The essential catalytic residues of polypeptides of the Rhomboid family are highly conserved and equate to residues N169, G215, S217, H281, W151 and R152 of the Drosophila Rhomboid-1 sequence. The essential residues required for cleavage by Rhomboid are residues A141, S142, G143 and A144 of the Spitz sequence or their equivalent in other rhomboid ligands. Other important residues include residues A138 S139 and I140 of the Spitz sequence or their equivalent in other rhomboid ligands.

These parts or residues constituting the active region of the compound are known as its “pharmacophore”. The information provided herein regarding the pharmacophore of the Rhomboid family and its substrate allow their structures to be modelled according their physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modelling process.

In a variation of this approach, the three-dimensional structure of the Rhomboid polypeptide and its substrate TMD are modelled. This can be especially useful where the ligand and/or binding partner change conformation on binding, allowing the model to take account of this the design of the mimetic.

A template molecule is then selected onto which chemical groups which mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. Modified compounds found by this approach can then be screened to see whether they have the target property, or to what extent they exhibit it. For example, mimetics which model the three-dimensional conformation of the Rhomboid recognition domain of a rhomboid ligand (for example, Spitz residues 140-144: IASGA, or more preferably residues 138-144: ASIASGA) may be used to screen for a compound which binds and inhibits a Rhomboid polypeptide. Such mimetics may include peptide chloromethyl ketone analogues of the Rhomboid-binding domain of a rhomboid ligand, for example, a Spitz analogue comprising the IASGA or ASIASGA sequence.

Further optimisation or modification can then be carried out to arrive at one or more final compounds for in vivo or clinical testing.

A pharmaceutical composition comprising a rhomboid modulator, for example an enhancer or inhibitor, may be administered to individuals, for example for the treatment (which may include preventative treatment) of a pathogenic infection or a condition associated with or mediated by Rhomboid activity, for example a cardiovascular disorder, including disorders associated with blood coagulation, an inflammatory disorder, or a cancer condition.

Cardiovascular disorders include disorders such as cardiac myxoma, acute myocardial infarction, stroke, in particular hemorrhagic stroke, ischaemic (coronary) heart disease, atherosclerosis, myocardial ischaemia (angina) and disorders associated with blood coagulation such as cerebral thrombosis, cerebral embolism, coronary artery thrombolysis, arterial and pulmonary thrombosis and embolism, and various vascular disorders such as peripheral arterial obstruction, deep vein thrombosis, disseminated intravascular coagulation syndrome, thrombus formation after artificial blood vessel operation or after artificial valve replacement, re-occlusion and re-stricture after coronary artery by-pass operation, re-occlusion and re-stricture after PTCA (percutaneous transluminal coronary angioplasty) or PTCR (percutaneous transluminal coronary re-canalization) operation and thrombus formation at the time of extracorporeal circulation.

Inflammatory disorders include allergy, asthma, atopic dermatitis, Crohn's disease, Felty's syndrome, gingivitis, pelvic inflammatory disease, periodontitis, polymyositis/dermatomyositis, psoriasis, rheumatic fever, rheumatoid athritis, skin inflammatory diseases, spondylitis, systemic lupus erythematosus, ulcerative colitis, uveitis, vasculitis and inflammation caused by sepsis or ischaemia.

Cancer conditions include cancers, (e.g., histocytoma, glioma, glioblastoma, astrocyoma and osteoma) including lung cancer, small cell lung cancer, gastrointestinal cancer, bowel cancer, oral cancer, colon cancer, breast cancer, oesophageal cancer, ovarian carcinoma, prostate cancer, testicular cancer, liver cancer, kidney cancer, bladder cancer, pancreas cancer, skin cancer and brain cancer.

Other disorders mediated by rhomboid activity include diabetes, disorders of peripheral nervous system, pneumonia, adult respiratory distress syndrome, chronic renal failure and acute hepatic failure.

An aspect of the present invention provides a modulator, for example an inhibitor of Rhomboid protease activity, or composition comprising a said modulator, isolated and/or obtained by a method described herein. Modulators are described in more detail above.

Another aspect of the invention provides a chimeric polypeptide which is proteolytically cleavable by a Rhomboid polypeptide, said polypeptide comprising an a core domain which has a rhomboid cleavable TMD sequence linked to an heterogenous upstream tag sequence, the core domain sequence not being susceptible to cleavage by mammalian metalloproteases.

Chimeric substrate polypeptides are described in more detail above.

Another aspect of the invention provides a nucleic acid encoding a chimeric substrate polypeptide as described above.

Nucleic acid encoding a chimeric substrate polypeptide may be provided as part of a replicable vector, particularly any expression vector from which the encoded polypeptide can be expressed under appropriate conditions, and a host cell containing any such vector or nucleic acid. An expression vector in this context is a nucleic acid molecule including nucleic acid encoding a polypeptide of interest and appropriate regulatory sequences for expression of the polypeptide.

Suitable vectors can be chosen or constructed, containing appropriate regulatory sequences, including promoter sequences, terminator fragments, polyadenylation sequences, enhancer sequences, marker genes and other sequences as appropriate.

A nucleic acid construct which comprises a nucleic acid sequence encoding a chimeric substrate polypeptide, may include an inducible promoter operatively linked to the nucleic acid sequence. This allows control of expression, for example, in response to an applied stimulus.

The term “inducible” as applied to a promoter is well understood by those skilled in the art. In essence, expression under the control of an inducible promoter is “switched on” or increased in response to an applied stimulus (which may be generated within a cell or provided exogenously). The nature of the stimulus varies between promoters. Whatever the level of expression is in the absence of the stimulus, expression from any inducible promoter is increased in the presence of the correct stimulus. Cells may thus be pre-incubated with a test compound prior to the induction of rhomboid expression.

Many examples of inducible promoters will be known to those skilled in the art (e.g. Tet on/Tet off system, BD Biosciences).

Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds. John Wiley & Sons, 1992.

Systems for cloning and expression of polypeptides in a variety of different host cells are well-known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells and many others. A common, preferred bacterial host is E. coli.

The introduction of nucleic acid into a host cell, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

Marker genes such as antibiotic resistance or sensitivity genes may be used in identifying clones containing nucleic acid of interest, as is well known in the art.

The introduction may be followed by causing or allowing co-expression from the nucleic acid, e.g. by culturing host cells (which may include cells actually transformed although more likely the cells will be descendants of the transformed cells) under conditions for co-expression of the coding sequences, so that the encoded polypeptides are produced.

Another aspect of the present invention provides a host cell comprising nucleic acid encoding a chimeric substrate polypeptide, as described herein. A host cell may comprise an expressed membrane-bound chimeric substrate polypeptide as described herein.

The nucleic acid may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

A host cell as described above may further comprise a heterologous nucleic acid encoding a rhomboid polypeptide, as described above. Suitable rhomboid encoding nucleic acid may be comprised in an expression vector.

A host cell may thus comprise a heterologous Rhomboid polypeptide and a heterologous substrate polypeptide as described herein.

Other aspects of the invention relate to the use of such nucleic acids, vectors and host cells in a method of screening for rhomboid activity, for example in a method described herein.

Methods described herein may also be useful in isolating and/or purifying rhomboid cleavage products. A method for obtaining a cleavage product of a rhomboid polypeptide may comprise:

(a) contacting a rhomboid polypeptide and a substrate polypeptide in the presence of one or more non-rhomboid proteases,

wherein said substrate polypeptide comprises a core domain which has a rhomboid cleavable TMD sequence linked to a heterogenous upstream tag sequence, the core domain sequence not being susceptible to cleavage by the one or more non-rhomboid proteases, and;

(b) contacting said medium with a specific binding member which binds to said tag sequence, and

(c) isolating/purifying soluble polypeptide fragment bound to said specific binding member.

Following isolation, the fragment may be investigated further, for example, the fragment may be sequenced.

Suitable specific binding members include antibodies or, when a polyHis tag is used, NiNTA.

Aspects of the present invention will now be illustrated with reference to the accompanying figures described below and experimental exemplification, by way of example and not limitation.

The skilled person will understand that the invention may be carried out with various combinations and sub-combinations of the features described above, and all these combinations and sub-combinations, whether or not specifically described or exemplified, are encompassed by the invention.

Further aspects and embodiments will be apparent to those of ordinary skill in the art. All documents mentioned in this specification are hereby incorporated herein by reference.

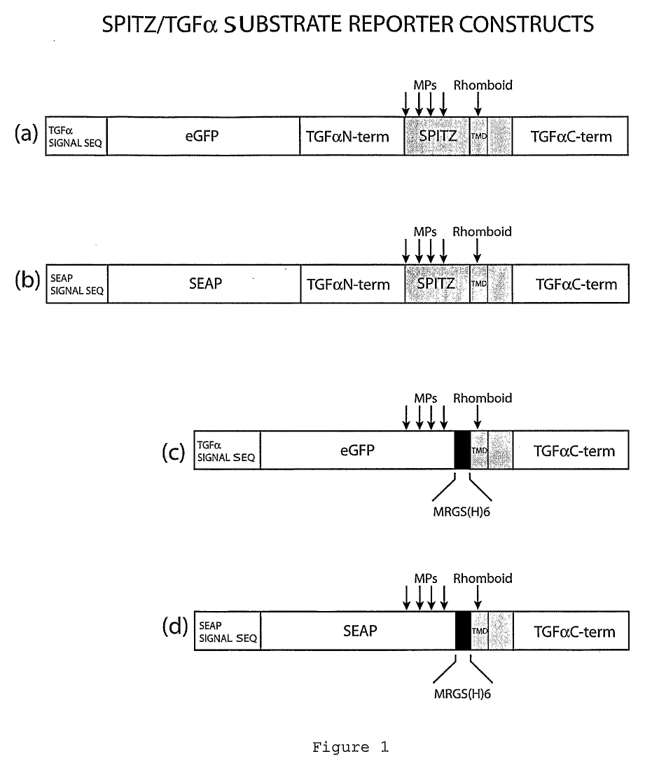

FIG. 1 shows reporter (substrate) constructs used for rhomboid assays: (a) GFP/TGFα/Spi/TGFα (b) SEAP/TGFα/Spi/TGFα (c) GFP/6H/Spi/TGFα (d) SEAP/6H/Spi/TGFα.

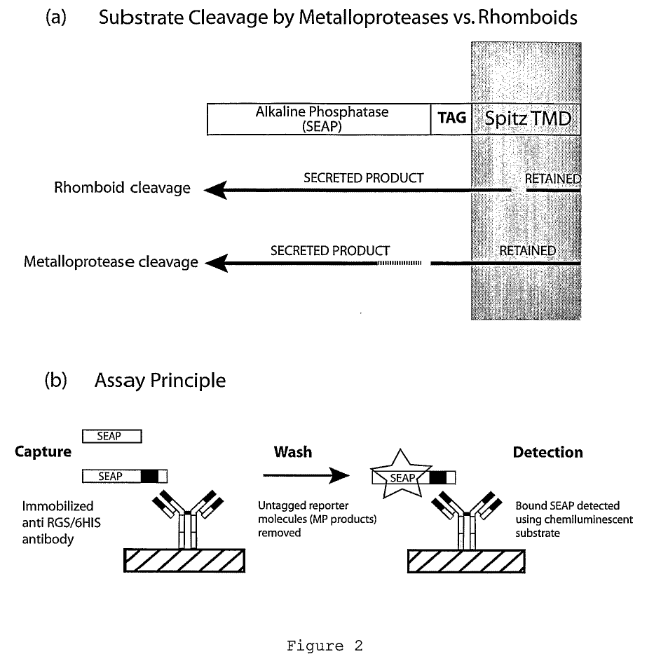

FIG. 2a illustrates the difference between the products generated by a rhomboid cleavage compared to those made by endogenous metalloproteases. The extracellular domain and SPITZ transmembrane domain of the substrate are shown and the location of the membrane is shaded.

FIG. 2b shows the principle of the capture assay.

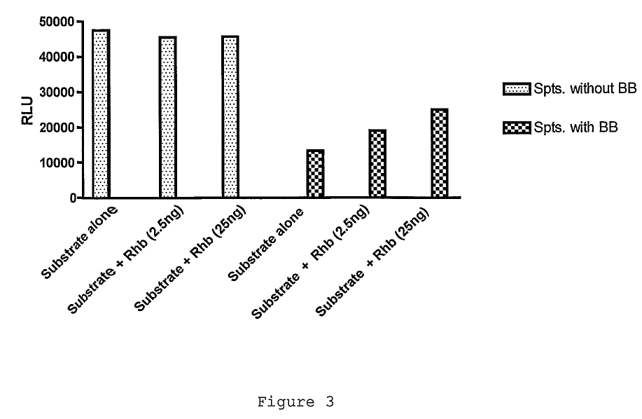

FIG. 3 shows total SEAP production in 48 h transfection supernatants performed in 6 well plates.

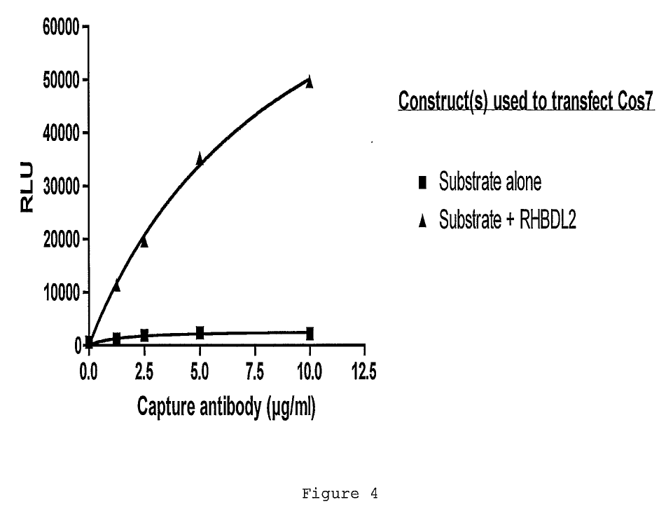

FIG. 4 shows the specific capture of rhomboid cleavage product using various concentrations of capture antibody.

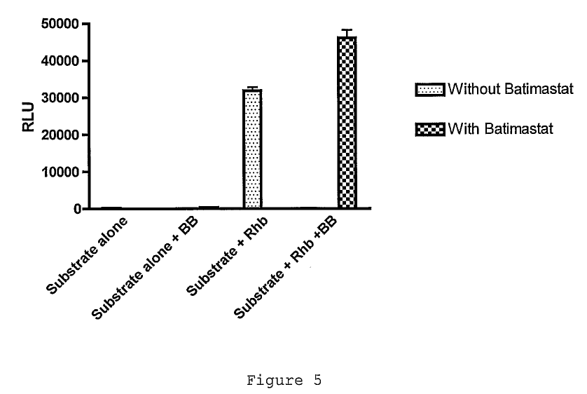

FIG. 5 shows capture assay results to show the selective capture of rhomboid/SEAP fusion products from large-scale transfection supernatants in the presence or absence of Batimastat (BB).

### EXAMPLES

### Materials and Methods

### Constructs

All constructs were generated in the vector pcDNA3.1 (Invitrogen). The construction of TGFα/SPITZ chimeras has been described previously (Urban & Freeman, 2003). The chimera GFP/TGFα/Spi/TGFα (construct a; FIG. 1) consists of GFP fused to the sequence encoding the first 51 amino acids of human TGFα, Drosphila SPITZ (aa 119-160) and human TGFα C-terminal region (aa 122-160).

To replace the GFP reporter and TGFα signal sequences with SEAP, the SEAP gene and signal sequence was amplified by PCR using the primers “HindSEAP For” (5′-AAGCTTCACCATGCTGCTGCTGCTGCTGCTGCT-3′) and “Eco Back” (5′-ACGGAATTCTGTCTGCTCGAAGCGGCCGGC-3′) and pSEAP-2 template DNA (Clontech). The product was cloned into GFP/TGFα/Spi/TGFα using HindIII and EcoRI restriction sites to generate SEAP/TGFα/Spi/TGFα (construct b, FIG. 1).

To prepare the construct GFP/6H/Spi/TGFα (construct c, FIG. 1), PCR primers were designed to amplify the SPITZ TMD and to introduce the MRGS(H)₆ tag sequence immediately upstream. The primers “6HMRGS For” (5′-CGGAATTCATGAGAGGATCGCATCACCATCACCATCACGCGAGCATTGCCAGTGGAGCCA-3′) and “BBS Back” (5′-CTGCTATTGTCTTCCCAATCCT-3′) were used to PCR amplify the SPITZ TMD using SEAP/TGFα/Spi/TGFα as the template. The product was cloned into GFP/TGFα/Spi/TGFα using EcoRI and Bbs-I restriction sites.

To obtain SEAP/6H/Spi/TGFα (construct d, FIG. 1), the construct GFP/6H/Spi/TGFα was digested with EcoRI and RsrII and the fragment was cloned into SEAP/TGFα/Spi/TGFα using the same sites. In the constructs GFP/6H/Spi/TGFα (construct c, FIG. 1) and SEAP/6H/Spi/TGFα (construct c, FIG. 1) the extracellular domain of SPITZ has been deleted.

The construction of the expression vector for human Rhomboid RHBDL2 has been described (Urban et al., 2001).

All constructs were verified by sequencing.

Transfection of Cos-7 with Rhomboid and Substrate Reporter Constructs

COS-7 cells were grown in DMEM medium containing 10% foetal calf serum (FCS) and antibiotics in 175 cm² growth area flasks (T175, Sarstedt) in a humidified atmosphere at 37° C./5% CO₂. The cells were passaged when they reached approximately 80% confluency. Transfections were performed in 6-well plates (Costar), 75 cm² (T75) or T175 flasks (Sarstedt).

For transfection, the cells were trypsinized, counted and adjusted to 1.0×10⁵/ml. Six well plate wells, T75 or T175 flasks were seeded with 2 ml, 6 ml or 24 ml cell suspension respectively. After returning to the incubator for 16 h, the cells were transfected with substrate reporter construct alone or together with RHBDL2.

Various amounts of substrate and rhomboid construct DNA were used in transfections using FuGENE 6 transfection reagent (Roche). The total DNA was maintained at 1 μg for 6 well plate transfections, 2 μg for T75 or 4 μg for T175 flask transfections by inclusion of PUC18 DNA. For transfection of cells in T175 flasks with substrate construct alone, 2 μg of plasmid DNA was prepared and adjusted to a total of 4 μg with PUC18 in a volume of 10-15 μl H₂O per flask. For co-transfection with Rhomboid, 1.4 μg RHBDL2 was mixed with 2 μg substrate construct and adjusted to a total of 4 μg as above.

Prior to transfection, 16 μl FuGENE 6 was diluted into 784 μl serum-free DMEM and mixed with the prepared plasmid DNA. After incubating at RT for 40 min, the mixture was added drop wise to the flask and returned to the incubator for 5-6 h. Next the cells were trypsinized and re-seeded into 96-well flat-bottomed tissue culture plates at 1×10⁴/well in 100 μl volumes and returned to the incubator for 16-18 h.

For some experiments, the cells were maintained in flasks without re-seeding. At this time the cells were rinsed once by aspirating and filling the wells with PBS and then replacing the medium with 100 μl/well serum-free DMEM alone or containing 20 μM batimastat (British Biotechnology).

For flask cultures the cells were rinsed once with 40 ml PBS and the media replaced with 12 ml serum-free DMEM +/− batimastat. After incubating for 24 h, supernatants were collected and either assayed directly for total SEAP activity or following capture with RGSHis antibody.

### Assay for Total SEAP Reporter Activity

Total SEAP activity in the supernatants was assayed in white polystyrene 96-well flat-bottomed plates (Costar) using CSPD® chemiluminescent substrate (Phospha-Light™ assay system, Applied Biosystems) according to the manufacturer's instructions. Briefly, supernatant samples (12.5 μl) were diluted with 37.5 μl of dilution buffer in 0.5 ml eppendorf tubes and heated to 65° C. for 30 min to destroy endogenous alkaline phosphatase before adding to the wells. After 5 min at RT, 50 μl reaction buffer was added to all wells and 20 min later luminescence was measured using a microplate luminometer (BMG PolarStar).

### Assay for SEAP Reporter After Capture With Immobilised RGS6His Antibody

RGS6His monoclonal antibody (Qiagen) was diluted to 2.5 μg/ml in PBS and 50 μl/well used to coat white polystyrene 96-well plates (Nunc Maxisorp) overnight at 4° C. The plates were washed 3 times with PBS containing 0.1% Tween 20 (PBS/T) using an automated plate washer. To block the wells, plates were incubated with 100 μl/well PBS/T containing 5% non-fat skimmed milk powder (Marvel) for 2 h at RT. After 3 washes with PBS/T, 50 μl neat transfection, supernatant was added to each test well and incubated for 2 h at RT. The plates were washed 5 times and 50 μl dilution buffer (Phospha-Light™ assay system, Applied Biosystems) added to all wells, followed by 50 μl assay buffer. After 5 min, ⁵⁰ μl reaction buffer was added to all wells and 20 min later luminescence was measured using a microplate luminometer (BMG PolarStar).

### Western Blot for MRGS/6His Tagged Polypeptides

Transfection supernatants were analysed by western blot for polypeptides containing the RGS6His tag sequence. Supernatant samples were subjected to reducing SDS PAGE using a mini gel apparatus (Atto) and transferred to PVDF membranes (Millipore). The membranes were blocked with PBST containing 5% non-fat skimmed milk powder (Marvel) for 1 h at RT. After washing with PBST, the blots were probed with anti-RGS6His monoclonal antibody (Qiagen) diluted 1:2000 in PBST containing 2.5% Marvel for 1 h at RT. After further washes the blots were incubated with goat anti-mouse IgG (Fc portion) secondary antibody peroxidase conjugate (Jackson ImmunoResearch) diluted 1:25,000 for 1 h at RT. Finally the blots were washed and incubated with enhanced chemiluminescent substrate (ECL plus, Amersham) according to the manufacturer's instructions before exposure to hyperfilm ECL (Amersham) and development.

### Results

1. Rhomboid reporter assay based on total SEAP activity in transfection supernatants

A chimeric substrate polypeptide (SEAP/6H/Spi/TGFα; 200 ng) comprising a core domain having the TMD of Drosphila Spitz and an MRGS(H)₆ tag, an extracellular domain having a secreted alkaline phosphatase label and a cytoplasmic domain comprising the TGFα C terminal domain and cytoplasmic sequence from Drosophila Spitz (construct d, FIG. 1) was expressed in Cos-7 cells alone or with RHBDL2(Rhb, 25 ng or 2.5 ng), in the presence or absence of batimastat (BB).

FIG. 3 shows the total SEAP activity in flask transfection supernatants following transfection with substrate alone or with RHBDL2 in the absence or presence of the hydroxamate inhibitor Batimastat. In the absence of Batimastat, total SEAP activity was highest and unaffected by the inclusion of RHBDL2 DNA in the transfection. However, in the presence of Batimastat, total SEAP activity was reduced, providing indication that the substrate is susceptible to cleavage by endogenous metalloproteases.

Following co-transfection of the substrate with RHBDL2 in the presence of Batimastat, total SEAP activity was increased relative to substrate alone indicating rhomboid-specific cleavage. The ratio of chemiluminescence signal in the presence of RHBDL2 to signal for substrate alone was approximately 2:1 when 25 ng of RHBDL2 was used to transfect the cells. This example shows that for an assay based on total SEAP reporter activity in the supernatants, inclusion of Batimastat is important in order to suppress the background signal due to metalloprotease-mediated substrate cleavage.

2. Rhomboid reporter assay based on captured SEAP activity in transfection supernatants

The SEAP/6H/Spi/TGFα substrate was designed to be cleaved by RHBDL2 or other rhomboids to release a secreted product that has the tag sequence at or near its C terminus (FIGS. 1 and 2). In contrast, following metalloprotease cleavage the tagged portion is retained in the membrane so the secreted product lacks the tag.

An assay was designed to selectively capture and measure the rhomboid cleavage product in the medium. In this assay, rhomboid products, which retain the tag, are captured with immobilized tag-specific antibodies. Following washing to remove untagged reporter products, captured reporter is assayed using a chemiluminescent substrate for SEAP (FIG. 2).

To this end, ELISA plates were coated with various concentrations of anti-RGS6His monoclonal antibody and incubated with flask transfection supernatants from SEAP/6H/Spi/TGFα substrate construct alone (as a control) or with RHBDL2 (FIG. 4).

Transfections were performed in T75 flasks using 400 ng substrate construct alone or with 100 ng rhomboid. The medium was supplemented with 20 μg/ml Batimastat and harvested at 48 h post-transfection.

After washing the plates to remove unbound reporter products, the retained reporter (SEAP) activity was assayed as described above. The results show that in the presence of RHBDL2, SEAP activity was detected in the wells following capture by the anti-RGS6His antibody. The measured activity was dose-dependent with respect to the antibody concentration used to coat the wells and fell to background levels in uncoated control wells. In contrast, only background reporter signal was detectable at any coating concentration for the control transfection supernatant (substrate alone). The ratio of chemiluminescence signal in the presence of rhomboid to that for the substrate alone was maximal at the highest coating concentration of antibody attempted.

Cos-7 cells were grown in T175 flasks and transfected with 2 μg of SEAP/6H/Spi/TGFα substrate construct alone or co-transfected with 1.4 μg rhomboid RHBDL2 (Rhb). The transfected cells were re-seeded into 96-well plates to enable the assay to be used as a high throughput screen for small molecule inhibitors of rhomboid and incubated +/− BB for a further 24 h before the supernatants were harvested and tested in the capture assay. In this format, supernatants from 100 μl cultures were assayed for SEAP after capture with anti-RGS6His-coated ELISA plates (FIG. 5). Transfected cells were incubated with or without Batimastat for comparison. The results show a signal to background ratio of 133:1 (substrate with rhomboid:substrate alone) in the presence of Batimastat and 129:1 without. Therefore a greatly improved assay performance was obtained. Furthermore, the results show that proteolytic cleavage events due to rhomboid may be assayed in the absence of metalloprotease inhibitors and in a high throughput format. A large reduction in the background signal was attained in the capture assay in comparison to the total SEAP reporter assay and rhomboid-specific cleavage product was specifically determined in the absence of suppression of endogenous metalloproteases.

3. Western Blotting with Anti-RGSHis Antibody

In order to determine the relative sizes of the tagged polypeptides, transfection supernatants were subjected to reducing SDS PAGE followed by transfer to PVDF membranes and western blot with the anti-RGS6His antibody. Cos-7 cells were transfected with SEAP/6H/Spi/TGFα alone or with rhomboid RHBDL2 in T175 flasks before being reseeded into 96 well plates. At 48 h post transfection, supernatants were harvested and tested in the western blot procedure for the presence of the tagged substrate product. The results show that a tagged product of approximately 70 kDa was present in the supernatants following transfection with rhomboid and substrate together, but not with substrate alone. The size and quantity of the tagged product was not apparently affected by the presence of batimastat in the transfection cultures. Total SEAP activity in the supernatants at the time of harvesting for the substrate alone and substrate plus RHBDL2 transfections were comparable (45561 RLU and 38251 RLU). This provides indication that the substrate alone transfection supernatants contain similar levels of active SEAP reporter product to those with rhomboid, but that rhomboid is necessary to produce the tagged reporter product.

Therefore the western blot results show that metalloproteases cleave the substrate upstream of the tag sequence to generate secreted reporter products that lack the tag sequence. In contrast, tagged products of the expected size were generated in the presence of rhomboid that may also be selectively assayed in the capture assay.

### 4. Rhomboid Capture Assay Evaluation Screen

A total of 11, 040 compounds including 10,000 synthetic small molecules (Maybridge, Tintagel, Cornwall, UK) and 1,040 purified natural products (Molecular Nature Ltd., Aberystwyth, UK) were screened in the Rhomboid capture assay using an automated liquid handling procedure. The compounds were contacted with the cells at a final concentration of 5 μM for 24 hours. An overall hit rate of 1.2% was obtained for inhibitors using a SEAP signal cut-off set at <3 SD of the mean of the negative control wells. Negative controls consisted of supernatants from wells containing cells transfected with human RHBDL2 and substrate constructs in the presence of an equivalent concentration of DMSO to that introduced by addition of a compound. Hits were identified and re-tested using the compound master stocks and gave a hit confirmation rate of 65%.

The assay was also found to be suitable for the identification of positive modulators of Rhomboid. These were observed at an overall frequency of 3.2%, of which 61% were confirmed in repeats using compound master stocks.

The potency of hits was ranked by testing serial dilutions of active compounds in the same assay (IC50 determination). IC50 determinations resulted in the identification of 9 inhibitors and 3 positive modulators with potencies of <10 μM.

TABLE 1

| Accession | Gene | Size | Species |
|---|---|---|---|
| P20350 | Rhomboid-1 | | Drosophila Melanogaster |
| AAK06753 | Rhomboid-3 | | Drosophila Melanogaster |
| AAK06752 | Rhomboid-2 | | Drosophila Melanogaster |
| CAA76629(XM_007948, | Rhomboid related | 438 | Homo Sapiens |
| NM_003961, AJ272344) | protein (RHBL) | | |
| | (GI:3287191) | | |
| AAK06754 | Rhomboid-4 | | Drosophila Melanogaster |
| NP_060291 | FLJ20435(GI:8923409) | 292 | Homo Sapiens |
| T16172 | F26F4.3 | 419 | C. elegans |
| AAA02747 | AAA02747 | 325 | Saccharum hybrid cultivar H65-7052 |
| S40723 | Rhomboid homlog | 397 | C. elegans |
| | C489B4.2 | | |
| AAF88090 | C025417_18 | 302 | Arabidopsis thaliana |
| AAG51610 | C010795_14 | 317 | Arabidopsis thaliana |
| AAD55606 | C008016_16 | 309 | Arabidopsis thaliana |
| CAB88340 | CAB8830 | 361 | Arabidopsis thaliana |
| AAG28519 | PARL (GI:11066250) | 379 | Homo sapiens |
| AE003628 | CG5364/Rhomboid-5 | 1840 | Drosophila melanogaster |
| CAB87281 | CAB87281 | 346 | Arabidopsis thaliana |
| T36724 | T36724 | 297 | Streptomyces coelicolor |
| A55862 | AarA | 281 | Providencia stuartii |
| BAA12519 | YpgP | 507 | B. subtilis |
| AAF53172 | CG17212/Rhomboid-6 | 263 | Drosophila melanogaster |
| BAB05140 | BH1421 | 514 | Bacillus halodurans |
| T02735 | T9I4.13 | 372 | Arabidopsis thaliana |
| CAA17304 | Rv0110 | 249 | Mycobacterium tuberculosis |
| T34718 | T34718 | 383 | Streptomyces coelicolor |
| BAB21138 | BAB21138 | 393 | Oryza sativa |
| AAD36164 | E001768_13 | 222 | Thermatoga maritime |
| AAD35669 | AE001733_6 | 235 | Thermatoga maritime |
| T35521 | T33521 | 256 | Streptomyces coelicolor |
| CAC18292 | CAC18292 | 497 | Neurospora crassa |
| T05139 | F7H19.260 | 313 | Arabidopsis thaliana |
| AAG40087 | AC079374_1 | 369 | Arabidopsis thaliana |
| B75109 | PAB1920 | 212 | Pyrococcus abyssi |
| AAK04268 | AE006254_9 | 230 | Lactococcus lactis |
| CAA76716 | CAA76716 | 164 | Rattus norvegicus |
| AAF58598 | CG8972/Rhomboid-7 | 351 | Drosophila melanogaster |
| CAA86933 | CAA86933 | 276 | Acinetobacter calcoaceticus |
| CAA97104 | YGR101w/Yeast | 346 | Saccharomyces cerevisiae |
| | Rhomboid-1 | | |
| AAC07308 | AAC07308 | 227 | Aquifex aeolicus |
| E72574 | APE1877 | 256 | Aeropyrum pernix |
| NP_069844 | NP_069844 | 330 | Archaeoglobus fulgibus |
| AAA58222 | AAA58222 | 274 | E. coli |
| BVECGG | GlpG | 276 | E. coli |
| E71025 | PH1497 | 197 | Pyrococcus horikoshii |
| AAK03522 | GlpG | 291 | Pasteurella multocida |
| G82780 | XF0649 | 224 | Xylella fastidiosa |
| G69772 | YdcA | 199 | Bacillus subtilis |
| O14362 | C30D10.19C | 298 | Schizosaccharomyces pombe |
| F82729 | XF1054 | 232 | Xylella fastidiosa |
| BAB04236 | BH0517 | 248 | Bacillus halodurans |
| T34866 | T34866 | 285 | Streptomyces coelicolor |
| A82363 | GlpG | 277 | Vibrio cholerae |
| I64081 | GlpG | 192 | Haemophilus influenzae |
| AC026238 | AC026238 | 336 | Arabidopsis thaliana |
| AAH03653 | AAH03653(GI:13177766) | 329 | Homo sapiens |
| D71258 | GlpG | 208 | Treponema pallidum |
| CAB9075 | CAB9075 | 223 | Streptococcus uberis |
| AAK24595 | AAK24595 | 218 | Caulobacter crescentus |
| B83259 | PA3086 | 286 | Pseudomonas aeruginosa |
| C82588 | XF2186 | 206 | Xylella fastidosa |
| AAG19304 | Vng0858c | 598 | Halobacterium sp. NRC-1 |
| BAB02051 | MKP6.17 | 506 | Arabidopsis thaliana |
| AAG18926 | Vng0361c | 333 | Halobacterium sp. NRC-1 |
| BAB29735 | BAB29735 | 315 | Mus musculus |
| E75328 | E75328 | 232 | Deinococcus radiodurans |
| T49293 | T16L24.70 | 269 | Arabidopsis thaliana |
| CAB83168 | CAB83168 | 392 | Schizosaccharomyces pombe |
| T45666 | F14P22.50 | 411 | Arabidopsis thaliana |
| P53426 | B1549_C3_240 | 251 | Mycobacterium leprae |
| CAC22904I | CAC22904I | 214 | Sulfolobus solfataricus |
| T41608 | SPCC790.03 | 248 | Schizosaccharomyces pombe |
| H81375 | Cj1003c | 172 | Campylobacter jejuni |
| CAC31552 | CAC31552 | 238 | Mycobacterium leprae |
| Q10647 | YD37_MYCTU | 240 | Mycobacterium tuberculosis |
| NP_015078 | Ypl246cp | 262 | Saccharomyces cerevisae |
| S76748 | S76748 | 198 | Synechocystis sp. |
| NM_017821 | RHBDL2 (GI:8923409) | | Homo sapiens |
| BE778475 | RHBDL3 (GI:10199673) | | Homo sapiens |

TABLE 2

| Accession | Name | Size | Species |
|---|---|---|---|
| Q01083 | Spitz (GI:50403762) | 230 | D. melanogaster |
| AAF63381 | Keren/Gritz/Spitz-2 | 217 | D. melanogaster |
| | GI:7533127 | | |
| P42287 | Gurken | 294 | D. melanogaster |
| | (GI:27808655) | | |
| P01135 | TGF-α (GI:135689) | 160 | Homo sapiens |
| P00533 | EGF (GI:2811086) | 1210 | Homo sapiens |
| Q99075 | HB-EGF (GI:544477) | 208 | Homo sapiens |
| JC1467 | Betacellulin | 178 | Homo sapiens |
| | (GI:345766) | | |
| A34702 | Amphiregulin | 252 | Homo sapiens |
| | (GI:107391) | | |
| BAA22146 | Epiregulin | 169 | Homo sapiens |
| | (GI:2381481) | | |
| Q03345 | Lin-3 (GI:417248) | 438 | C. elegans |

1. A method for identifying and/or obtaining a modulator of a rhomboid polypeptiide, which method comprises:
(a) contacting a rhomboid polypeptide and a substrate polypeptide in the presence of a test compound and one or more non-rhomboid proteases,
wherein said substrate polypeptide comprises a core domain which has a rhomboid cleavable TMD sequence linked to an upstream tag sequence, the core domain sequence not being susceptible to cleavage by the one or more non-rhomboid proteases, and;
(b,) determining the presence or amount in said medium of a soluble polypeptide fragment comprising said tag sequence.

2. A method according to claim 1 wherein said Rhomboid polypeptide and said substrate polypeptide are co-expressed in a cell.

3. A method according to claim 2 wherein the cell is a mammalian cell.

4. A method according to claim 1 wherein the presence of the soluble substrate polypeptide is determined by;
(a) contacting said medium with an specific binding member which binds to said tag sequence, and
(b) determining binding of soluble polypeptide fragment to said binding member.

5. A method according to claim 4 wherein said specific binding member is immobilised.

6. A method according to claim 5 wherein said specific binding member is an antibody.

7. A method according to claim 6 wherein said antibody is immobilised on the surface of microtitre plate.

8. A method according to claim 1 wherein the substrate polypeptide comprises an extracellular detectable label.

9. A method according to claim 8 wherein the label is secreted alkaline phosphatase.

10. A method according to claim 8 wherein the binding of said polypeptide fragment to said anti-tag antibody is detected by determining the amount of said label bound to the antibody.

11. A method according to claim 10 wherein the amount of said label is determined by contacting said label with a reporter molecule which produces a signal in the presence of said label, and measuring said signal.

12. A method according to claim 11 wherein the signal is light emission.

13. A method according to claim 1 wherein the tag sequence is positioned 10 amino acid residues or less upstream of said TMD in said core domain.

14. A method according to claim 1 wherein the tag sequence consists of 30 amino acids or less.

15. A method according to claim 1 wherein the tag sequence is MRGS(H)₆.

16. A method according claim 1 wherein the rhomboid cleavage TMD rhomboid comprises a lumenal portion which has the same conformation within the membrane as Spitz residues 140-144.

17. A method according to claim 16 wherein the rhomboid cleavable TMD has a lumenal portion which comprises or consists of Spitz residues 140-144 (IASGA).

18. A method according to claim 16 wherein the rhomboid cleavable TMD is a rhomboid ligand TMD.

19. A method according to claim 18 wherein the rhomboid cleavable TMD is the Spitz TMD.

20. A method according to claim 1 wherein the substrate polypeptide comprises a cytoplasmic domain, said domain comprising the cytoplasmic domain of TGFα.

21. A method according to claim 1 wherein the substrate polypeptide comprises a cytoplasmic domain, said domain comprising the cytoplasmic domain of thrombomodulin.

22. A method according to claim 1 wherein the Rhomboid polypeptide has a sequence shown in Table 1.

23. A method according to claim 22 wherein the Rhomboid polypeptide is selected from the group consisting of Drosophila Rhomboid 1, Drosophila Rhomboid 2, Drosophila Rhomboid 3, Drosophila Rhomboid 4, Human RHBDL-1, Human RHBDL-2 and Human RHBDL-3, E. coli gIgG, B. subtilis ypqP, P. stuartii A55862 gene product, P. aeruginosa B83259 gene product, S. cerevisiae YGRIOIw and S. cerevisiae YPL246c.

24. A method according to claim 1 comprising identifying said test compound as a modulator of Rhomboid protease activity.

25. A method according to claim 24 comprising isolating said test compound.

26. A method according to claim 25 comprising synthesising and/or preparing said test compound.

27. A method according to claim 25 comprising modifying said compound to optimise the pharmaceutical properties thereof.

28. A method according to claim 24 comprising formulating said test compound in a pharmaceutical composition with a pharmaceutically acceptable excipient, vehicle or carrier.

29. A modulator of Rhomboid protease activity obtained by a method of claim 1.

30. A method of making a pharmaceutical composition comprising,
identifying a compound as a modulator of Rhomboid activity the method according to claim 1,
synthesising, preparing or isolating said compound and admixing the compound with a pharmaceutically acceptable excipient, vehicle or carrier, and optionally other ingredients to formulate or produce said composition.

31. A method according to claim 30 comprising modifying said compound to optimise the pharmaceutical properties thereof.

32. A method according to claim 30 comprising determining the activity of a Rhomboid polypeptide in the presence of said composition.

33. A polypeptide which is proteolytically cleavable by a Rhomboid polypeptide, said polypeptide comprising an a core domain which has a rhomboid cleavable TMD sequence linked to an upstream tag sequence, the core domain sequence not being susceptible to cleavage by mammalian metalloproteases.

34. A polypeptide according to claim 33 wherein the tag sequence is positioned 10 amino acid residues or less upstream of said TMD in said core domain.

35. A polypeptide according to claim 33 wherein the tag sequence consists of 15 amino acids or less.

36. A polypeptide according to claim 35 wherein the tag sequence is MRGS(H)₆.

37. A polypeptide according claim 33 wherein the rhomboid cleavage TMD rhomboid comprises a lumenal portion which has the same conformation within the membrane as Spitz residues 140-144.

38. A polypeptide according to claim 37 wherein the rhomboid cleavable TMD has a lumenal portion which comprises or consists of Spitz residues 140-144 (IASGA).

39. A polypeptide according to claim 37 wherein the rhomboid cleavable TMD is a rhomboid ligand TMD.

40. A polypeptide according to claim 39 wherein the rhomboid cleavable TMD is the Spitz TMD.

41. A polypeptide according to claim 33 wherein the substrate polypeptide comprises an extracellular domain, said domain comprising a detectable label.

42. A polypeptide according to claim 41 wherein the label is secreted alkaline phosphatase.

43. A polypeptide according to claim 33 wherein the substrate polypeptide comprises a cytoplasmic domain, said domain comprising the cytoplasmic domain of thrombomodulin.

44. An isolated nucleic acid encoding a chimeric polypeptide according to claim 33.

45. An expression vector comprising a nucleic acid according to claim 44.

46. A host cell comprising an expression vector according to claim 45.

47. A host cell according to claim 46 further comprising an expression vector comprising a nucleic acid encoding a rhomboid polypeptide.

48. A method for obtaining a cleavage product of a Rhomboid polypeptide, which method comprises:
(a) contacting a Rhomboid polypeptide and a substrate polypeptide and one or more non-rhomboid proteases, wherein said substrate polypeptide comprises a core domain which has a rhomboid cleavable TMD sequence linked to an upstream tag sequence, the core domain sequence not being susceptible to cleavage by the one or more non-rhomboid proteases, and;
(b) contacting said medium with an antibody which binds to said tag sequence, and
(c) isolating/purifying soluble polypeptide fragment bound to said antibody.

49. A method according to claim 48 comprising sequencing the polypeptide fragment.


SEQUENCE LISTING (15 sequences)

SEQ ID NO: 1 - protein, 10 aa, Artificial sequence
  source: /note= “Description of artificial sequence Tag sequence”
MRGSHHHHHH

SEQ ID NO: 2 - protein, 8 aa, Artificial sequence
  source: /note= “Description of artificial sequence Tag sequence”
DYKDDDDK

SEQ ID NO: 3 - protein, 15 aa, Artificial sequence
  source: /note= “Description of artificial sequence Tag sequence”
KETAAAKFERQHMDS

SEQ ID NO: 4 - protein, 8 aa, Artificial sequence
  source: /note= “Description of artificial sequence Tag sequence”
WSHPQFEK

SEQ ID NO: 5 - protein, 10 aa, Artificial sequence
  source: /note= “Description of artificial sequence Tag sequence”
EQKLISEEDL

SEQ ID NO: 6 - protein, 12 aa, Artificial sequence
  source: /note= “Description of artificial sequence Tag sequence”
MKAEFRRQESDR

SEQ ID NO: 7 - protein, 12 aa, Artificial sequence
  source: /note= “Description of artificial sequence Tag sequence”
MRDALDRLDRLA

SEQ ID NO: 8 - protein, 5 aa, Drosophila melanogaster
IASGA

SEQ ID NO: 9 - protein, 7 aa, Drosophila melanogaster
ASIASGA

SEQ ID NO: 10 - protein, 4 aa, Artificial sequence
  source: /note= “Description of artificial sequence Endoplasmic reticulum retention signal”
KDEL

SEQ ID NO: 11 - protein, 4 aa, Drosophila melanogaster
ASGA

SEQ ID NO: 12 - DNA, 33 bp, Artificial sequence
  source: /note= “Description of artificial sequence Primer HindSEAP For”
aagcttcaccatgctgctgctgctgctgctgct

SEQ ID NO: 13 - DNA, 30 bp, Artificial sequence
  source: /note= “Description of artificial sequence Primer Eco Back”
acggaattctgtctgctcgaagcggccggc

SEQ ID NO: 14 - DNA, 60 bp, Artificial sequence
  source: /note= “Description of artificial sequence Primer 6HMRGS For”
cggaattcatgagaggatcgcatcaccatcaccatcacgcgagcattgccagtggagcca

SEQ ID NO: 15 - DNA, 22 bp, Artificial sequence
  source: /note= “Description of artificial sequence Primer BBS Back”
ctgctattgtcttcccaatcct